(19) United States
(12) Patent Application Publication
(10) Pub. No.: US 2004/0022911 A1
(43) Pub. Date: Feb. 5, 2004

(54) Method of sustaining alertness

(76) Inventors: Luc Gruffaz, Richmond (CA); Philippe Lavoie, Victoria (CA); Darren Drew, Edmonton (CA); Nina Hirva, Vancouver (CA); Brita Moosmann, Vancouver (CA)

Correspondence Address: KOLISCH HARTWELL, P.C., 520 S.W. YAMHILL STREET, SUITE 200, PORTLAND, OR, 97204, US

(21) Appl. No.: 10/412,473

(22) Filed: Apr. 11, 2003

Related U.S. Application Data
(60) Provisional application No. 60/371,410, filed on Apr. 11, 2002.

(51) Int. Cl. A61K 47/00; A23L 1/00

(52) U.S. Cl. 426/531; 424/439

(57) ABSTRACT

A method of sustaining alertness of attendees during a meeting is provided, comprising the steps of (a) providing at least a meal from a meal plan to said attendees, each of said meals comprising at least a food and at least a fluid, said meal plan configured to enhance energy levels by selection of a quantity of each of said foods for said meal based on a nutritional value of said food; and a quantity of each of said fluids for said meal plan based on a nutritional value of said fluid; (b) providing a time schedule configured to promote attentiveness by providing break periods wherein foods and fluids from said meal plan are served to said attendees; and (c) providing an environment configured to promote concentration by manipulating atmospheric conditions of a meeting space wherein said meeting takes place.

This application claims priority from U.S. Provisional Application Serial No. 60/371,410 filed on Apr. 11, 2002 by Gruffaz et al., and the entire disclosure of such provisional application is expressly incorporated herein by reference.

### NOTICE REGARDING COPYRIGHTED MATERIAL

A portion of the disclosure of this patent document contains material which is subject to copyright protection. The copyright owner has no objection to the facsimile reproduction by anyone of the patent document or the patent disclosure as it appears in the public Patent Office file or records but otherwise reserves all copyright rights whatsoever.

### TECHNICAL FIELD

This invention relates to a method of sustaining alertness, more particularly, a method of sustaining alertness during a meeting.

### BACKGROUND

Meetings, conferences, seminars, workshops, and other similar events (all of which are referred to herein as “meetings”) are regularly held for business, educational and networking purposes. Facilities such as conference centers and hotels typically provide meeting space for such events and meeting managers are responsible for planning and executing the meeting. The duties of meeting managers typically include: planning the agenda, setting a budget, selecting a meeting place, and negotiating the services to be provided for the meeting by a meeting space provider and/or other service suppliers and contractors. Regardless of how well planned a meeting is, there is a tendency for attention to wander as attendees start losing concentration and fatigue sets in. Physical factors of the environment and nutritional content of meals are two main factors that affect the alertness of attendees during meetings.

Most meeting spaces are multi-purpose and therefore not designed to provide an optimal learning environment. For example, meeting spaces provided by hotels are typically also used for banquets and other social functions. As such, physical factors of the environment conducive to learning and alertness are often overlooked.

Productivity and the learning process are also influenced by the comfort provided by the atmosphere of the meeting space. Lighting that is too bright tires the eyes and dim lighting induces sleep. Temperature that is too warm decreases concentration level and fosters day dreaming and temperature that is too cool causes discomfort. Poor indoor air quality, such as airborne dust and pollens that irritate the respiratory system and undesirable smells can be distracting. Low humidity levels dehydrate attendees and affect brain function. In order to facilitate the continued participation and attention of attendees and to assist such attendees in staying focussed throughout the meeting, a physical environment conducive to learning should be provided.

Food is known to have an effect on concentration, mood, and cognition. Often restaurant or other prepared meals served during meetings are higher in fat and calories than what is eaten on a regular basis, which can potentially have a negative effect on productivity. Poor energy levels and reduced performance have been observed in a number of instances where meal content was either high in fat or high in total calories. For example, higher fat breakfasts have been associated with a depressed alertness, less vigour and imagination and more fatigue whereas lower fat breakfasts have been associated with higher post-breakfast alertness. With respect to meal portions, eating large quantities often lead to reduced concentration and post-prandial drowsiness.

Although most food and beverage providers for meetings are aware of common dietary restrictions (e.g. vegetarians and religious restrictions) and the general trend of health conscious eating (e.g. low-fat, high protein meals), meal plans are typically not prepared to ensure proper nutritional balance or to promote concentration and alertness. For example, most meals and foods provided during break periods are presented buffet style without recommendations on size of serving.

Consuming healthy servings of whole grains, vegetables and leaner protein foods limits the amount of fat and total calories in meals and helps provide the nutritional balance needed to promote optimal health, and improve alertness, concentration, and post-prandial state. “Light” products and leaner protein foods reduce the amount of fat and total calories in meals. Higher fibre grains provide a more sustained release of energy to fuel the body over time. Fruits and vegetables provide nutrition and volume to reduce the amount of calorie-dense foods required. Smaller meal portions reduce the metabolic demands on the body (i.e. to produce insulin and to digest the meal), thereby leaving more energy for other tasks (e.g. concentration and learning). Fluids are also essential to maximize the central nervous system.

### SUMMARY OF THE INVENTION

It is an object of the present invention to provide a method of sustaining alertness for a meeting to optimize learning and productivity by providing a well-balanced nutritional meal plan that enhances the energy levels of attendees of the meeting; by providing an environment whereby the temperature, air quality, and lighting is manipulated to reduce environmental distractions and promote optimal concentration of attendees of the meeting; and by providing a time schedule for meals and refreshments to promote attentiveness.

A method of sustaining alertness of attendees during a meeting is provided, comprising the steps of: (a) providing at least a meal from a meal plan to said attendees, each of said meals comprising at least a food and at least a fluid, said meal plan configured to enhance energy levels by selection of a quantity of each of said foods for said meal based on a nutritional value of said food; and a quantity of each of said fluids for said meal plan based on a nutritional value of said fluid; (b) providing a time schedule configured to promote attentiveness by providing break periods wherein foods and fluids from said meal plan are served to said attendees; and (c) providing an environment configured to promote concentration by manipulating atmospheric conditions of a meeting space wherein said meeting takes place.

### BRIEF DESCRIPTION OF FIGURES

Further objects, features and advantages of the present invention will become more readily apparent to those skilled in the art from the following description of the invention when taken in conjunction with the accompanying drawings, in which:

FIG. 1 shows three sample dinner meal plans according to the invention;

FIGS. 2 and 2A show four sample meal plans according to the invention, each including two meals and two break periods;

FIGS. 3 through 6 are charts showing samples of servings of grains, fruits and vegetables, milks, and meats, respectively; and

FIGS. 7 through 10 are samples of cards to be placed by the foods and fluids available to attendees.

### DETAILED DESCRIPTION OF PREFERRED EMBODIMENTS

Throughout this document, the term “attendees” means individuals attending a meeting.

Meal Plan

In an embodiment of the present invention, a meal plan designed to balance nutritional value and quantity of food, and type and quantity of fluids is provided. Such meal plan is configured to enhance energy levels thereby promoting post-prandial alertness, concentration, and optimal health.

The meal plan is designed for multi-day meetings which include three meals (i.e. breakfast, lunch, and dinner) and two break periods (i.e. morning and afternoon break periods) each day. The meal plan may be provided for any number of days with the option of including or excluding dinner on the last day. The meal plan typically serves between 20-60 attendees.

Nutritional Value and Quantity of Food

The plan uses food groups as defined by Canada's Food Guide to Healthy Eating (published by Health Canada, 1997, and incorporated herein by reference). The meal plan includes foods from the following food groups (provided with their portions):

(1) Grain products, preferably whole grains (e.g. whole wheat, oats, barley and rye) and enriched products (e.g. enriched pasta, ready-to-eat bran cereals or oatmeal, and multi-grain breads): between 5-12 servings a day. Sample grain servings are one slice of bread or ½ cup rice or pasta;

(2) Vegetables and fruits, preferably dark green and orange vegetables (e.g. spinach, broccoli, yams, and carrots) and orange fruit (e.g. oranges and cantaloupes): between 5-10 servings a day. Sample fruit and vegetable servings are one medium piece of fruit or ½ cup of cut fruit;

Milk products, preferably lower fat milk products (e.g. skim milk and low fat yoghurt): between 2-4 servings a day. Sample milk servings are 1 cup milk, 50 grams cheese or ¾ cup yoghurt; and

Meats and alternatives, preferably, leaner meats, fish, poultry, as well as beans and lentils: between 2-3 servings a day. Sample meat servings are 50 grams (cooked) meat, 1 egg or ⅓ cup tofu.

In an embodiment of the invention, whole grain foods are provided throughout the day whenever possible, including products such as multi-grain breads at breakfast and enriched pasta at lunch and/or dinner. Typically, fruits and vegetables are provided throughout the day and preferably, a fruit basket is available in the meeting space at all times. Milk products, which typically include fruit yoghurt and low-fat cream cheese, are provided at breakfast. Meats and other protein alternatives, typically including turkey, beef, and fish, are provided at lunch and/or dinner.

The average daily intake of 2500-2700 total calories a day is adequate for most people to maintain their body weight. The quantity of food for the meal plan which provides food for two meals (e.g. breakfast and lunch) and two break periods (e.g. a morning and an afternoon break period) per day is designed to provide a total caloric value of 1700-2000 calories per attendee. Alternatively, the quantity of food for the meal plan which provides a full day of food (i.e. three meals and two break periods) is designed to provide a total caloric value of 2500-2700 calories per attendee. Typically, the total calories of each meal and each break period is as follows:

(1) Breakfast: between 500-550 calories.

(2) Morning break period: between 250-300 calories.

(3) Lunch: between 650-700 calories.

(4) Afternoon break period: between 250-300 calories.

(5) Dinner: between 750-800 calories.

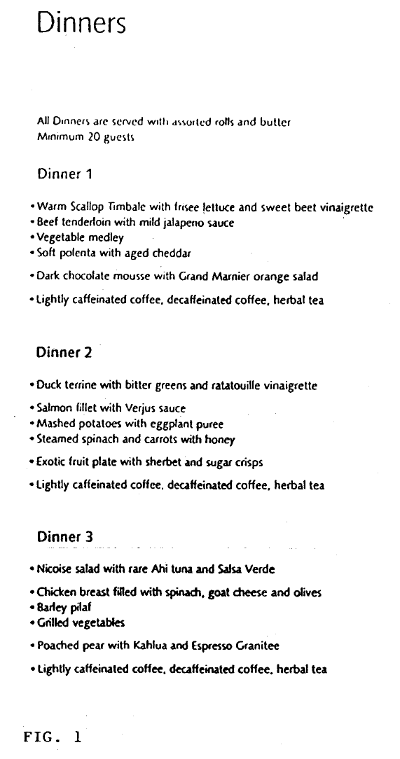

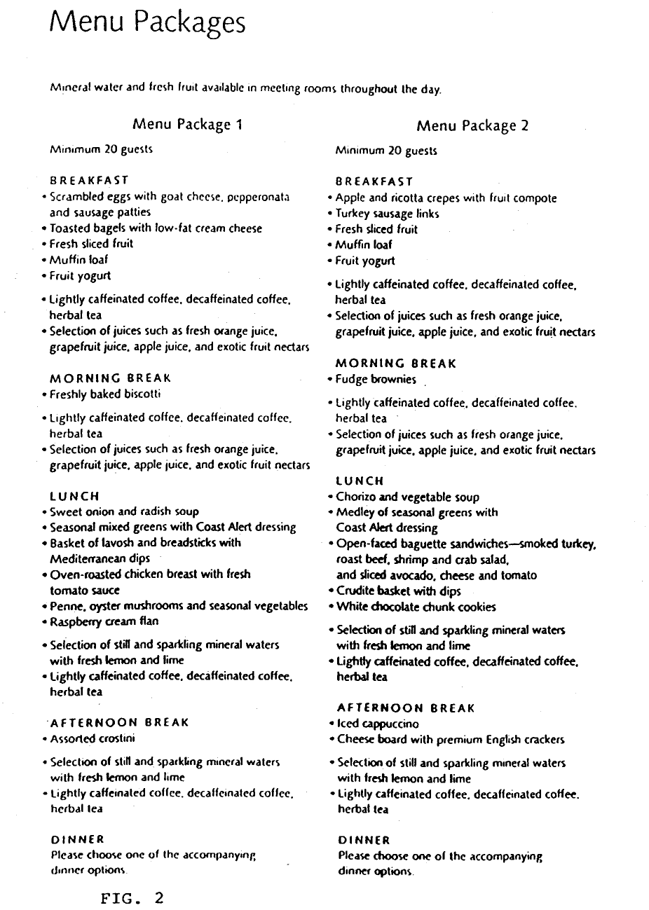

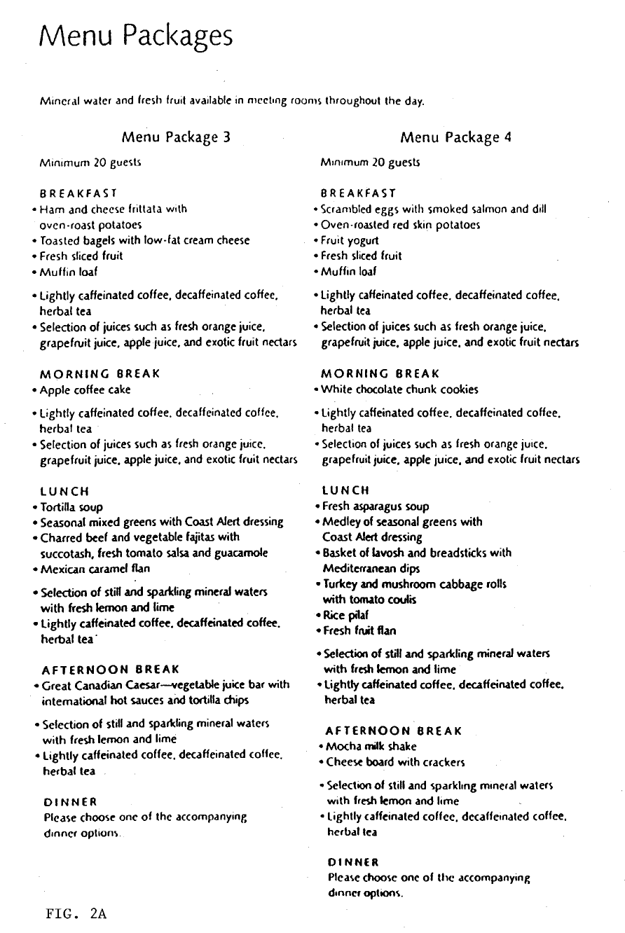

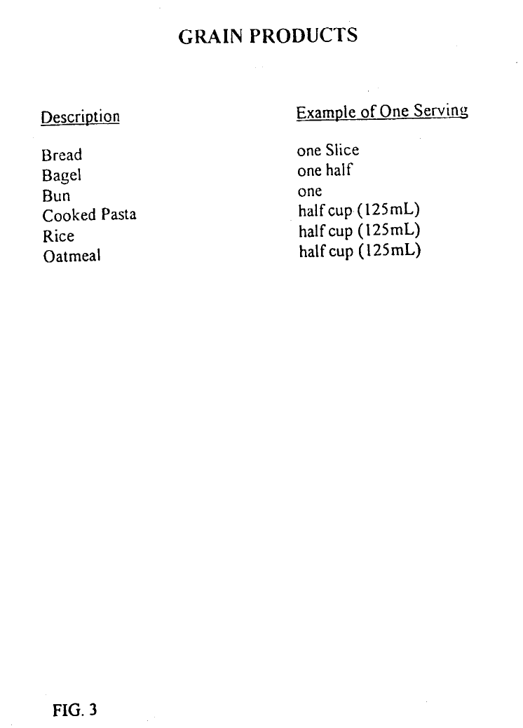

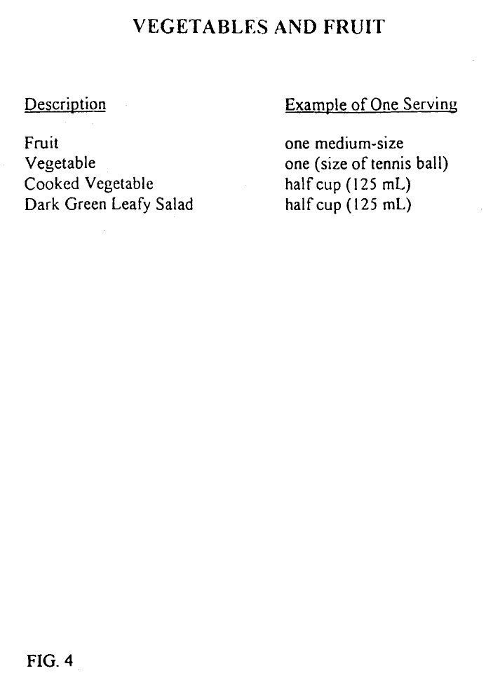

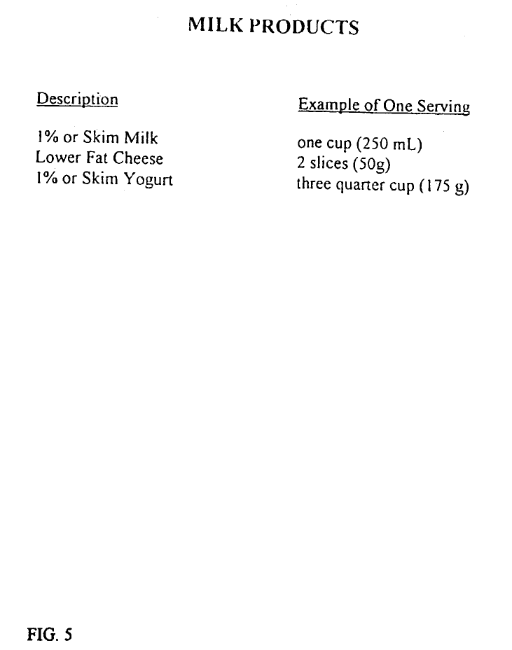

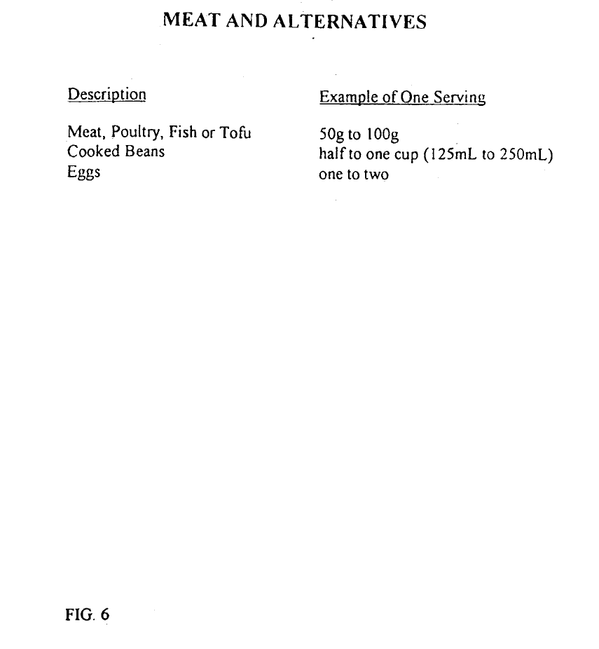

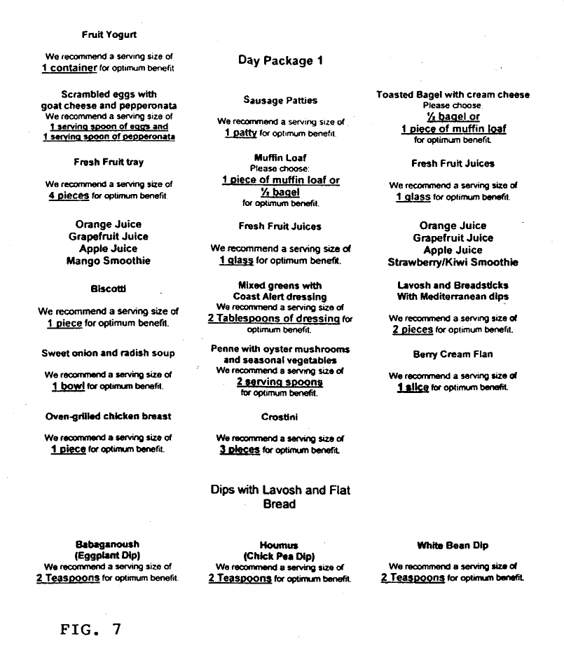

FIG. 1 sets out a sample of foods from the meal plan for two meals and two break periods and FIG. 2 sets out a sample of foods from the meal plan for dinner.

The main sources of calories from the meal plan include carbohydrates, fats, and proteins. In an embodiment of the invention, carbohydrates are between 45%-55% of total calories, fats are between 25%-30% of total calories, and proteins are between 15%-20% of total calories. Total carbohydrate content of the meal plan usually includes breads, rice, pasta, and potatoes. Total fat content of the meal plan is limited to healthy levels, especially with respect to saturated fats. Typically, fat is limited to approximately 30% of total calories wherein saturated fats constitute less than 10% of total calories. Vegetable oils, such as olive and canola, are used in food preparation. The use of animal fats is limited. Total protein content of the meal plan is mainly derived from leaner cuts of meat products (e.g. chicken breast) and fish and seafood. Foods that are higher in sugar and fat including desserts and salad dressings are a part of the meal plan but are provided in moderation.

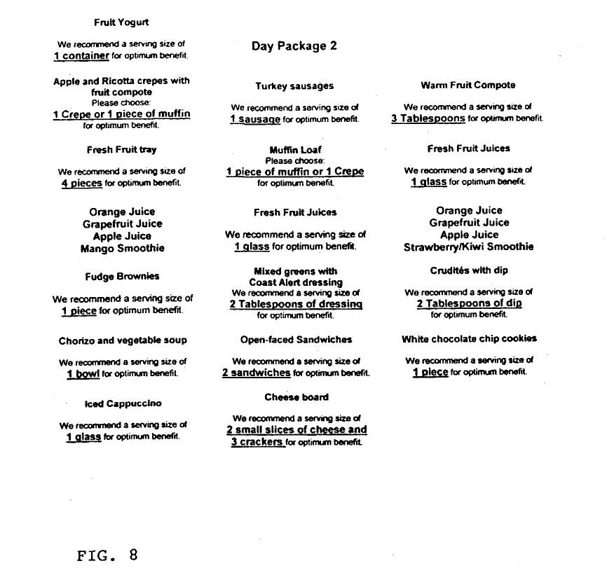

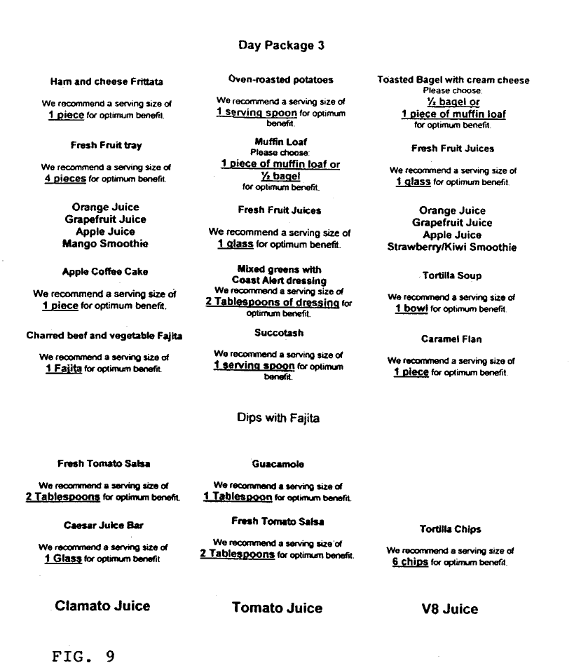

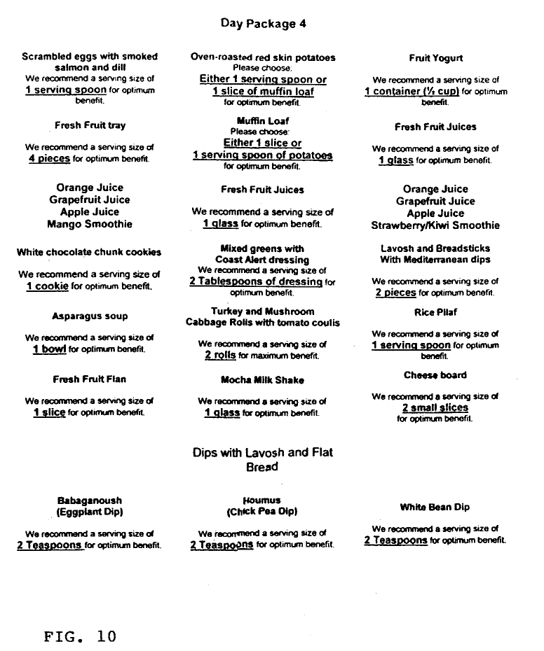

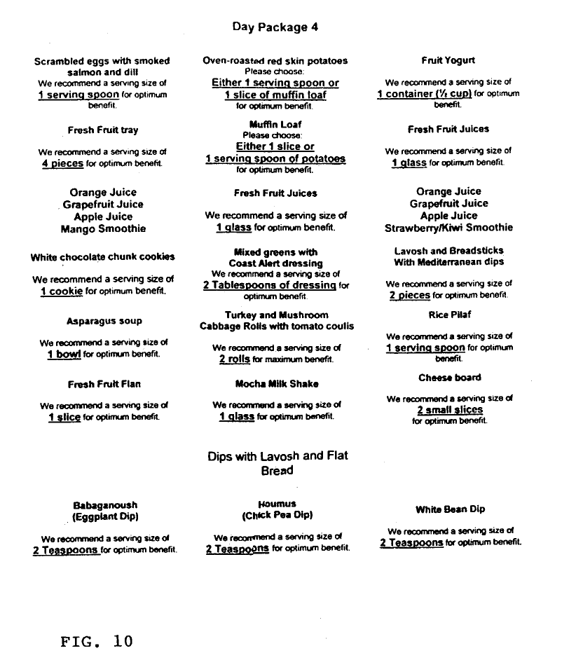

In an embodiment of the present invention, the recommended quantity of food per attendee is based on the total caloric needs an average adult requires as set out above, as seen in FIGS. 7 to 10. Typically, food from the meal plan is served buffet style with small signs indicating the type of food (e.g. oven-grilled chicken breast or penne with oyster mushrooms and seasonal vegetables) and the recommended quantity of food (e.g. one piece or two serving spoons) based on the desired total caloric value per day set out above. The signs are preferably printed on standard shipping labels which are 1″ by 2⅝″ and placed on tent cards, but any display means may be used as long as the printed matter is clearly visible.

In an alternative embodiment, a chart of the major allergens and items suitable for common restricted diets is provided. For example, foods suitable for vegans and vegetarians are listed, and foods containing peanuts are identified for attendees who have a peanut allergy.

In another alternative embodiment, any higher fat and sugar foods are removed from the in-room snack baskets if attendees stay overnight at a hotel. Suitable foods to be placed in the snack basket for attendees include pretzels, trail mix, nuts, dried fruit, and granola bars (not chocolate covered).

Fluids

Hydrating fluids are provided to assist the central nervous system (i.e. brain function). Hydrating fluids, including water, herbal tea, and 100% fruit juices, are provided instead of dehydrating fluids such as alcohol, caffeine-containing drinks, and high sugar drinks. Alternatively, lightly caffeinated and decaffeinated coffee are provided with a sign recommending decaffeinated coffee to the attendees. Preferably, alcohol is not provided as a part of the meal plan. However, if attendees do consume alcohol, it is recommended that no more than one to two glasses of wine or beer are consumed and that the alcohol is consumed only with dinner.

In an alternative embodiment, all alcoholic beverages, pop, and other dehydrating fluids are removed from the mini bar if attendees stay overnight at a hotel. Suitable fluids to be placed in the mini bar for attendees include water, juice, and natural juice spritzers.

Time Schedule

In an embodiment of the invention, a breakfast period, a lunch period and a dinner period is provided during which meals from the meal plan are served. Preferably, two break periods are provided throughout a day (e.g. morning break period and afternoon break period) and foods and fluids from the meal plan are provided during such break periods. The typical duration of a break period is 10 minutes. Preferably, a break period is provided every 50 minutes, but is typically provided between 1 hour and 50 minutes to 2 hours, as, for most attendees, attention starts to wane after 1 hour and 50 minutes.

In an embodiment of the invention, breakfast is served at 7:00 a.m. and the meeting commences at 8:00 a.m. The morning break period is at 10:00 a.m. and lunch is provided at 12:00 p.m. The meeting recommences at 1:00 p.m., the afternoon break is at 2:30 p.m. and dinner is served at 5:00 p.m.

Environment

In an embodiment of the invention, atmospheric conditions of the environment of the meeting space, including arrangement of furniture, temperature, air quality, and lighting are manipulated to promote concentration. The temperature is maintained between 18° C. and 23° C. (and ideally between 19° C. and 21° C.). Preferably, ventilation, air filtration, and humidity-management systems are provided. Typically, two High Efficiency Particulate Arrestance (“HEPA”) filters and two humidifiers are placed in the meeting space. The meeting room should be illuminated to at least between 20-50 foot candles of light. Other modifications to the environment could include aromatherapy or sounds (such as classical music or nature sounds).

The combination of the above elements presented to attendees of the meeting, including the quality and amount of food served, the environment of the meeting, and the timing of breaks, have a desirable effect of making the attendees considerably more alert and productive than has been the case for meetings.

While the principles of the invention have now been made clear in the illustrated embodiments, it will be immediately obvious to those skilled in the art that many modifications may be made of structure, arrangements, and algorithms used in the practice of the invention, and otherwise, which are particularly adapted for specific environments and operational requirements, without departing from those principles. The claims are therefore intended to cover and embrace such modifications within the limits only of the true spirit and scope of the invention.

What is claimed is:

1. A method of sustaining alertness of attendees during a meeting, comprising the steps of:
(a) providing at least a meal from a meal plan to said attendees, each of said meals comprising at least a food and at least a fluid, said meal plan configured to enhance energy levels by selection of:
(i) a quantity of each of said foods for said meal based on a nutritional value of said food; and
(ii) a quantity of each of said fluids for said meal plan based on a nutritional value of said fluid;
(b) providing a time schedule configured to promote attentiveness by providing break periods wherein foods and fluids from said meal plan are served to said attendees; and
(c) providing an environment configured to promote concentration by manipulating atmospheric conditions of a meeting space wherein said meeting takes place.

2. The method of claim 1 wherein said quantity of food of said meal plan is configured to provide a total caloric value between 1700 to 2000 calories a day to each of said attendees.

3. The method of claim 1 wherein said quantity of food of said meal plan is configured to provide a total caloric value between 2500 to 2700 calories a day to each of said attendees.

4. The method of claim 2 wherein said total caloric value comprises:
(a) between 45% to 55% carbohydrates;
(b) between 25% to 30% fats; and
(c) between 15% to 20% protein.

5. The method of claim 4 wherein said nutritional value of said meals in said meal plan provided to said attendee each day comprises:
(a) 5 to 12 servings of grain products;
(b) 5 to 10 servings of fruits and vegetables;
(c) 2 to 4 servings of milk products; and
(d) 2 to 3 servings of meat products.

6. The method of claim 5 wherein said fluids comprise selections of:
(a) water;
(b) 100% fruit juices;
(c) decaffeinated coffee; and
(d) herbal tea.

7. The method of claim 2 wherein said time schedule comprises:
(a) a breakfast period;
(b) a morning break period;
(c) a lunch period; and
(d) an afternoon break period.

8. The method of claim 3 wherein said time schedule comprises:
(a) a breakfast period;
(b) a morning break period;
(c) a lunch period;
(d) an afternoon break period; and
(e) a dinner period.

9. The method of claim 7 wherein each of said periods commence between 1 hour and 50 minutes to 2 hours after each of said previous periods end.

10. The method of claim 9 wherein said atmospheric conditions of said meeting space comprise:
(a) maintaining temperature of said meeting space between 18° C. and 23° C.;
(b) maintaining air quality in said meeting space by providing ventilation, air filtration, and humidity management systems; and
(c) illuminating said meeting room to at least between 20 and 50 foot candles of light.

11. The method of claim 10 wherein classical music is played to said attendees during said periods.

12. The method of claim 11 wherein aromatherapy is provided to said attendees during said periods.
